(19) United States
(12) Patent Application Publication
(10) Pub. No.: US 2012/0259225 A1
(43) Pub. Date: Oct. 11, 2012

(54) ULTRASOUND DIAGNOSTIC APPARATUS AND ULTRASOUND IMAGE PRODUCING METHOD

(75) Inventor: Rika TASHIRO, Kanagawa (JP)

(73) Assignee: FUJIFILM CORPORATION, Tokyo (JP)

(21) Appl. No.: 13/437,266

(22) Filed: Apr. 2, 2012

(30) Foreign Application Priority Data
Apr. 8, 2011 (JP) 2011-086198

(51) Int. Cl. A61B 8/14

(52) U.S. Cl. 600/443

(57) ABSTRACT

An ultrasound diagnostic apparatus includes a region of interest setting unit for setting a region of interest containing at least part of a liver and at least part of a kidney in a B mode image, a controller for transmitting and receiving ultrasonic beams to obtain reception data for measuring a sound speed, a sound speed map producer for producing a sound speed map of a liver region based on the reception data for measuring a sound speed, and a hepatorenal contrast calculator for calculating an average luminance in the liver region and an average luminance in a kidney region of the region of interest and calculating a hepatorenal contrast based on the calculated average luminances.

### BACKGROUND OF THE INVENTION

The present invention relates to an ultrasound diagnostic apparatus and an ultrasound image producing method and particularly to an ultrasound diagnostic apparatus that produces a B mode image by transmitting and receiving ultrasonic waves through a transducer array of an ultrasound probe.

Conventionally, ultrasound diagnostic apparatus using ultrasound images are employed in medicine. In general, this type of ultrasound diagnostic apparatus comprises an ultrasound probe having a built-in transducer array and an apparatus body connected to the ultrasound probe. The ultrasound probe transmits an ultrasonic beam toward the inside of a subject's body, receives ultrasonic echoes from the subject, and the apparatus body electrically processes the reception signals to produce an ultrasound image.

In diagnosis of fatty liver or like disease, use is often made of the ratio of average liver luminance to kidney average luminance or a so-called hepatorenal contrast as findings among other ultrasound findings. A fatty liver tends to increase the hepatorenal ratio contrast.

JP 2011-5026 A, for example, proposes an ultrasound diagnostic apparatus that performs hepatoscopy by measuring a hepatorenal contrast based on a liver image contained in a B mode image.

### SUMMARY OF THE INVENTION

The apparatus described in JP 2011-5026 A enables diagnosis using not only a B mode image but a measured hepatorenal contrast.

However, the hepatorenal contrast is known to be increased by, for example, chronic hepatitis as well as a fatty liver, making it difficult in some cases to give a high accuracy diagnosis based solely on a B mode image and a hepatorenal contrast.

An object of the present invention is to provide an ultrasound diagnostic apparatus and an ultrasound image producing method that resolve such problems in the art and enable a high accuracy diagnosis of a subject's liver disease.

An ultrasound diagnostic apparatus according to the present invention comprises:

a transducer array;

a transmission circuit for transmitting an ultrasonic beam from the transducer array toward a subject;

a reception circuit for processing reception signals outputted from the transducer array having received ultrasonic echoes from the subject to obtain reception data;

an image producer for producing a B mode image based on the reception data for a B mode image obtained by the reception circuit;

a monitor for displaying a B mode image produced by the image producer;

a region of interest setting unit for setting a region of interest containing at least part of a liver and at least part of a kidney of the subject in the B mode image displayed on the monitor;

a controller for controlling the transmission circuit and the reception circuit to transmit and receive ultrasonic beams with transmission focuses formed at a plurality of points set in at least a liver of the region of interest to obtain reception data for measuring a sound speed on at least the liver region of the region of interest;

a sound speed map producer for producing a sound speed map of the liver region of the region of interest based on the reception data for measuring a sound speed; and

a hepatorenal contrast calculator for calculating an average luminance in the liver region and an average luminance in the kidney region of the region of interest and calculating a hepatorenal contrast based on the calculated average luminances,

wherein the controller causes the monitor to display the sound speed map produced by the sound map producer and the hepatorenal contrast calculated by the hepatorenal contrast calculator.

An ultrasound image producing method comprises the steps of:

producing a B mode image based on reception data for a B mode image obtained by transmitting and receiving ultrasonic beams through a transducer array to and from a subject to display the B mode image on a monitor;

setting a region of interest containing at least part of a liver and at least part of a kidney of the subject in the B mode image;

transmitting and receiving ultrasonic beams with transmission focuses formed at a plurality of points set in at least a liver region of the region of interest to obtain reception data for measuring a sound speed on at least the liver region of the region of interest;

producing a sound speed map of the liver region of the region of interest based on the reception data for measuring a sound speed;

calculating an average luminance in the liver region and an average luminance in the kidney region of the region of interest and calculating a hepatorenal contrast based on the calculated average luminances; and

displaying the produced sound speed map and the calculated hepatorenal contrast on the monitor.

### BRIEF DESCRIPTION OF THE DRAWINGS

FIG. 1 is a block diagram illustrating a configuration of an ultrasound diagnostic apparatus according to Embodiment 1 of the invention.

FIGS. 2A and 2B schematically illustrate a principle of sound speed calculation in Embodiment 1.

FIG. 3 illustrates a B mode image where a region of interest and a plurality of lattice points are set in Embodiment 1.

FIG. 4 illustrates the contents displayed by a monitor in Embodiment 1.

FIG. 5 illustrates a B mode image where a region of interest and a plurality of lattice points are set in Embodiment 2.

FIG. 6 illustrates how lattice points set on a B mode image are classified according to their regions.

FIG. 7 illustrates a B mode image where a region of interest and a plurality of lattice points are set in a modification of Embodiment 2.

FIG. 8 illustrates a B mode image where a region of interest and a plurality of lattice points are set in another modification of Embodiment 2.

### DETAILED DESCRIPTION OF THE INVENTION

Embodiments of the present invention will be described below based on the appended drawings.

### Embodiment 1

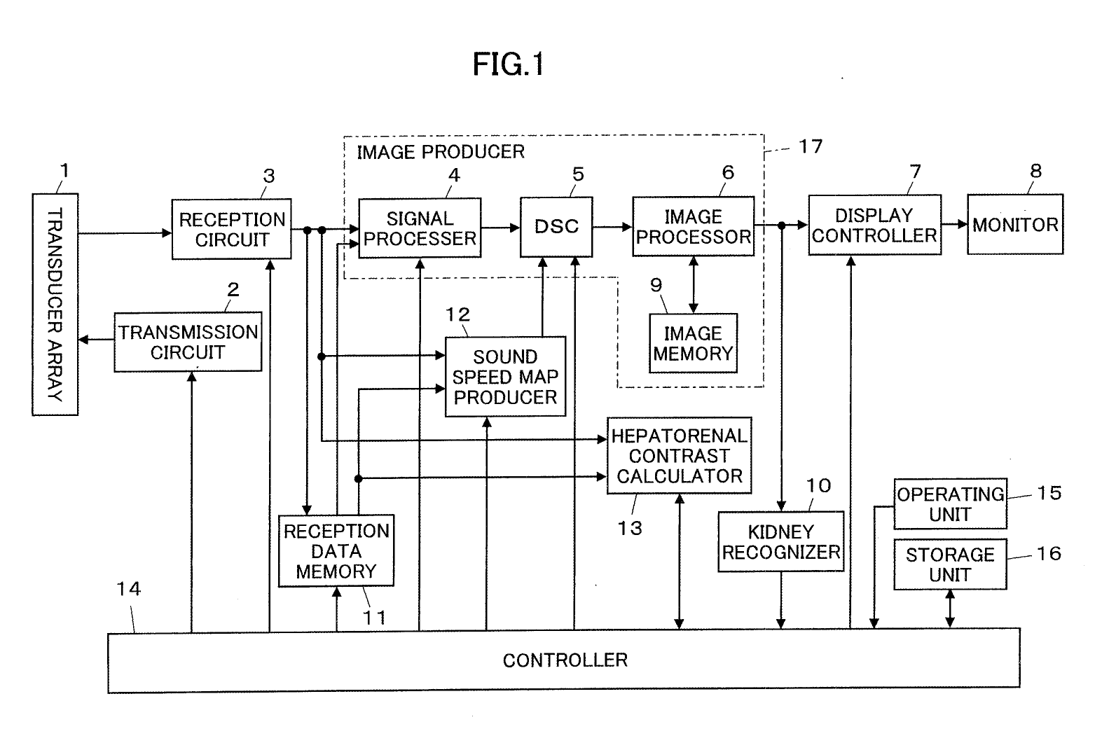

FIG. 1 illustrates a configuration of an ultrasound diagnostic apparatus according to Embodiment 1 of the invention. The ultrasound diagnostic apparatus of the invention comprises a transducer array 1, which is connected to a transmission circuit 2 and a reception circuit 3. A signal processor 4, a DSC (Digital Scan Converter) 5, an image processor 6, a display controller 7, and a monitor 8 are connected to the reception circuit 3. in sequences. An image memory 9 and a kidney recognizer 10 are connected to the image processor 6.

A reception data memory 11, a sound speed map producer 12 and a hepatorenal contrast calculator 13 are connected to the reception circuit 3. A controller 14 is connected to the transmission circuit 2, the reception circuit 3, the signal processor 4, the DSC 5, the display controller 7, the kidney recognizer 10, the reception data memory 11, the sound speed map producer 12 and the hepatorenal contrast calculator 13. An operating unit 15 and a storage unit 16 are further connected to the controller 14.

The transducer array 1 comprises a plurality of ultrasound transducers arranged one-dimensionally or two-dimensionally. These ultrasound transducers each transmit ultrasonic waves according to driving signals supplied from the transmission circuit 2 and receive ultrasonic echoes from the subject to output reception signals. Each of the ultrasound transducers comprises a vibrator composed of a piezoelectric body and electrodes each provided on both ends of the piezoelectric body. The piezoelectric body is composed of, for example, a piezoelectric ceramic represented by a PZT (titanate zirconate lead), a polymeric piezoelectric device represented by PVDF (polyvinylidene flouride), or a piezoelectric monochristal represented by PMN-PT (lead magnesium niobate lead titanate solid solution).

When the electrodes of each of the vibrators are supplied with a pulsed voltage or a continuous-wave voltage, the piezoelectric body expands and contracts to cause the vibrator to produce pulsed or continuous ultrasonic waves. These ultrasonic waves are combined to form an ultrasonic beam. Upon reception of propagating ultrasonic waves, each vibrator expands and contracts to produce an electric signal, which is then outputted as reception signal of the ultrasonic waves.

The transmission circuit 2 includes, for example, a plurality of pulse generators and adjusts the delay amounts for driving signals based on a transmission delay pattern selected according to an instruction signal transmitted from the transmission controller 14 so that the ultrasonic waves transmitted from a plurality of ultrasound transducers of the transducer array 1 form an ultrasonic beam and supplies the ultrasound transducers with delay-adjusted driving signals.

The reception circuit 3 amplifies and A/D-converts the reception signals transmitted from the ultrasound transducers of the transducer array 1, and then performs reception focusing processing by providing the reception signals with respective delays according to the sound speed or the sound speed distribution that is set based on a reception delay pattern selected according to the control signal from the controller 14 and adding them up. This reception focusing processing yields reception data (sound ray signals) having the ultrasonic echoes well focused.

The signal processor 4 corrects attenuation of the reception data produced by the reception circuit 3 according to the distance, i.e., the depth at which the ultrasonic waves are reflected, and then performs envelope detection processing to produce a B mode image signal, which is tomographic image information on a tissue inside the subject's body.

The DSC5 converts the B mode image signal produced by the signal processor 4 into an image signal compatible with an ordinary television signal scanning mode (raster conversion).

The image processor 6 performs various necessary processing including gradation processing on the B mode image signal entered from the DSC 5 before outputting the B mode image signal to the display controller 7 or storing the B mode image signal in the image memory 9.

The signal processor 4, the DSC 5, the image processor 6, and the image memory 9 constitute an image producer 17.

The display controller 7 causes the monitor 8 to display an ultrasound diagnostic image based on the B mode image signal having undergone image processing through the image processor 6.

The monitor 8 includes a display device such as an LCD, for example, and displays an ultrasound diagnostic image under the control of the display controller 7.

The kidney recognizer 10 detects the renal cortex of a kidney through, for example, pattern matching based on the B mode image signal having undergone image processing through the image processor 6.

The reception data memory 11 sequentially stores the reception data outputted from the reception circuit 3. The reception data memory 11 stores information on a frame rate entered from the controller 14 in association with the above reception data. Such frame rate information includes, for example, the depth of a position at which the ultrasonic waves are reflected, the density of scan lines, and a parameter representing the range of the visual field.

Under the control by the controller 14, the sound speed map producer 12 calculates the local sound speeds in a tissue inside the subject's body under examination based on the reception data stored in the reception data memory 11 to produce the sound speed map.

The hepatorenal contrast calculator 13 calculates an average luminance in the liver region and an average luminance in the kidney region based on the reception data for producing the B mode image produced by the reception circuit 3 and calculates a hepatorenal contrast based on these average luminances.

The controller 14 controls the components in the ultrasound diagnostic apparatus according to the instruction entered by the operator using the operating unit 15.

The operating unit 15 is provided for the operator to perform input operations and may be composed of, for example, a keyboard, a mouse, a track ball, and/or a touch panel. The operating unit 15 and the kidney recognizer 10 constitute a region of interest setting unit of the invention.

The storage unit 16 stores, for example, an operation program and may be constituted by, for example, a recording medium such as a hard disk, a flexible disk, an MO, an MT, a RAM, a CD-ROM, a DVD-ROM.

The signal processor 4, the DSC 5, the image processor 6, the display controller 7, the kidney recognizer 10, the sound speed map producer 13, and the hepatorenal contrast calculator 13 are each constituted by a CPU and an operation program for causing the CPU to perform various kinds of processing, but they may be each constituted by a digital circuit.

To display the B mode image, a plurality of ultrasound transducers of the transducer array 1 first transmit ultrasonic waves according to the driving signals supplied from the transmission circuit 2, and the ultrasound transducers having received ultrasonic echoes from the subject output the reception signals to the reception circuit 3, which produces the reception data. The signal processor 4, having received the reception data, produces the B mode image signal, the DSC 5 performs raster conversion of the B mode image signal, and the image processor 6 performs various image processing on the B mode image signal, whereupon, based on this B mode image signal, the display controller 7 causes the monitor 8 to display the ultrasound diagnostic image.

The local sound speed may be calculated by, for example, a method described in JP 2010-99452 A filed by the Applicant of the present application.

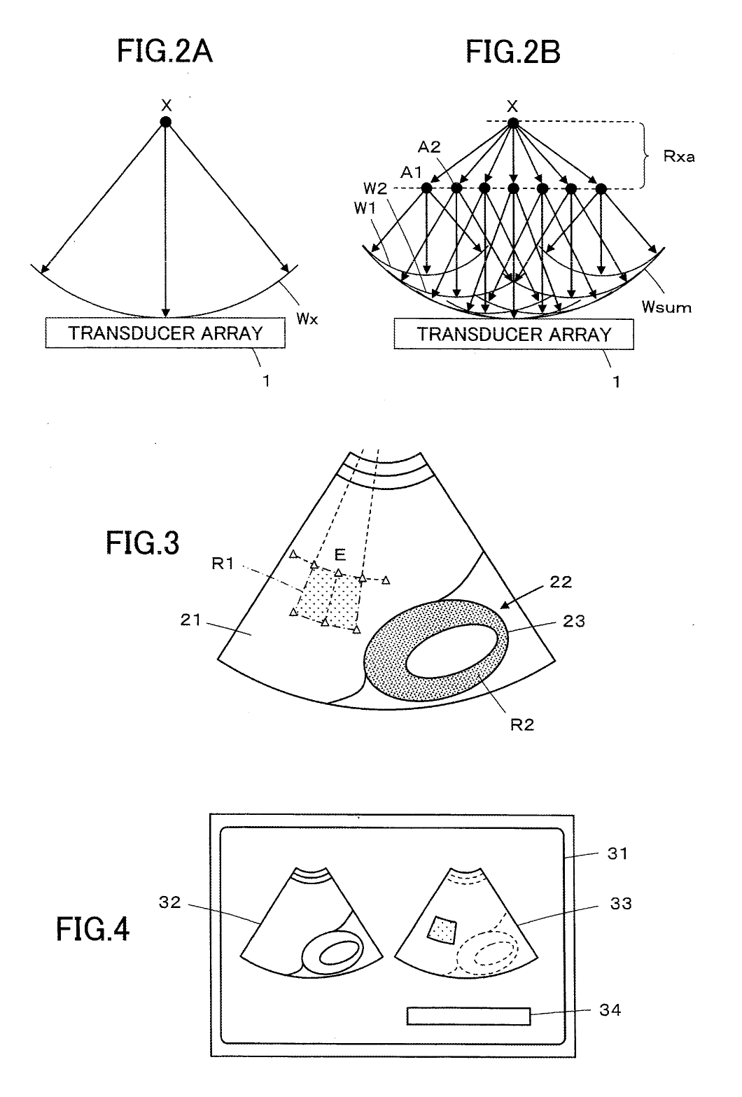

This method obtains the local sound speed at a lattice point X according to the Huygens principle. Suppose now that, on transmission of ultrasonic waves to the inside of a subject, a reception wave Wx reaches the transducer array 1 from the lattice point X, a reflection point in the subject, as illustrated in FIG. 2A, and that a plurality of lattice points A1, A2, . . . are arranged at equal intervals in positions shallower than the lattice point X, i.e., closer to the transducer array 1, as illustrated in FIG. 2B. Then, the local sound speed at the lattice point X is obtained according to the Huygens principle whereby a synthesized wave Wsum produced by combining reception waves W1, W2, . . . transmitted from the lattice points A1, A2, . . . having received a reception signal from the lattice point X coincides with the reception wave Wx from the lattice point X.

First, an optimum sound speed for all the lattice points X, A1, A2, . . . is obtained. The optimum sound speed herein means a sound speed allowing a highest image contrast and sharpness to be obtained as a set sound speed is varied after performing focus calculation for the lattice points based on the set sound speeds and imaging to produce an ultrasound image. The optimum sound speed may be judged based on, for example, the image contrast, spatial frequency in the scan direction, and dispersion as described in JP 08-317926 A.

Next, the optimum sound speed for the lattice point X is used to calculate the waveform of an imaginary reception wave Wx emitted from the lattice point X.

Further, a hypothetical local sound speed V at the lattice point X is changed to various values to calculate the imaginary synthesized wave Wsum of the reception waves W1, W2, . . . from the lattice points A1, A2, . . . Suppose that, at this time, the sound speed is consistent in a region Rxa between the lattice point X and the lattice points A1, A2, . . . and is equivalent to the local sound speed V at the lattice point X. The times in which the ultrasonic wave propagating from the lattice point X reaches the lattice points A1, A2, . . . are XA1/V, XA2/V, . . . , respectively, where XA1, XA2, . . . are the distances between the lattice point X and the lattice points A1, A2, . . . Combining the reflected waves emitted from the lattice points A1, A2, . . . with respective delays corresponding to the times XA1/V, XA2/V, . . . yields the imaginary synthesized wave Wsum.

Next, the respective differences between a plurality of the imaginary synthesized waves Wsum calculated by changing the hypothetical local sound speed V at the lattice point X to various values and the imaginary reception waves Wx from the lattice point X are calculated to determine the hypothetical local sound speed V at which the difference becomes a minimum as the local sound speed. The difference between the imaginary synthesized waves Wsum and the imaginary reception waves Wx from the lattice point X may be calculated by any of appropriate methods including a method using the cross-correlation, a method using phase matching addition by multiplying the reception waves Wx by a delay obtained from the synthesized wave Wsum, and a method using phase matching addition by multiplying the synthesized wave Wsum by a delay obtained from the reception waves Wx.

Thus, the local sound speeds inside a subject can be accurately calculated based on the reception data produced by the reception circuit 3. The sound speed map representing a distribution of the local sound speeds in a set region of interest may be likewise produced.

Next, the operation of Embodiment 1 will be described.

First, with the ultrasound probe placed by the operator on the subject's body surface close to the liver and a kidney, a plurality of ultrasound transducers of the transducer array 1 scans the whole region under observation with an ultrasonic beam they transmit according to the driving signals supplied from the transmission circuit 2, and the ultrasound transducers having received ultrasonic echoes from the subject output the reception signals to the reception circuit 3, which produces the reception data for the B mode image. The reception data are sequentially stored in the reception data memory 11 and outputted to the image producer 17 to produce a B mode image signal. Based on the B mode image signal, the display controller 7 causes the monitor 8 to display the B mode image.

Thus, as illustrated in FIG. 3, a B mode image containing at least part of a liver 21 and at least part of a kidney 22 is obtained.

In the process, the kidney recognizer 10 automatically recognizes a renal cortex 23 of the kidney 22 based on the B mode image signal produced by the image producer 17, and the recognition data are outputted to the controller 14.

As the operator operates the operating unit 15 to set a first region of interest R1 in the region of the liver 21 of the B mode image displayed on the monitor 8, the controller 14 sets a plurality of lattice points E indicated by “Δ” for measuring the sound speed in the first region of interest R1 and in its periphery. FIG. 3 illustrates the first region of interest R1 in the shape of a sector according to the convex display.

Subsequently, the controller 14 controls the transmission circuit 2 and the reception circuit 3 to transmit and receive the ultrasonic beam with the transmission focus formed at the lattice points E, and the reception data for measuring the sound speed produced by the reception circuit 3 for each ultrasonic beam received are sequentially stored in the reception data memory 11.

When the reception data for measuring the sound speed have been obtained for all the lattice points E, the controller 14 outputs an instruction for producing the sound speed map to the sound speed map producer 12, which then uses the reception data for measuring the sound speed stored in the reception data memory 11 to calculate local sound speeds at the lattice points E and produce the sound speed map of the inside of the first region of interest R1.

Further, as illustrated in FIG. 3, the controller 14 sets a second region of interest R2 in the region of the renal cortex 23 of the kidney 22 automatically recognized by the kidney recognizer 10. Upon receiving an instruction to calculate the hepatorenal contrast from the controller 14, the hepatorenal contrast calculator 13 calculates an average luminance B1 in the first region of interest R1 set in the liver 21 and an average luminance B2 in the second region of interest R2 set in the region of the renal cortex 23 based on the reception data for the B mode image stored in the reception data memory 11 and calculates the hepatorenal contrast expressed as r=B1/B2 or the ratio of the average luminance B1 in the first region of interest R1 to the average luminance B2 in the second region of interest R2.

The data of the sound speed map of the inside of the first region of interest R1 produced by the sound speed map producer 12 and hepatorenal contrast data on the hepatorenal contrast calculated by the hepatorenal contrast calculator 13 are sent to the DSC 5 under the control by the controller 14, undergo raster conversion through the DSC 5 as well as various image processing through the image processor 6 before being transmitted to the display controller 7. Subsequently, according to the display mode entered by the operator through the operating unit 15, the monitor 8 displays on its screen 31 a B mode image 32 and a sound speed map image 33 in juxtaposition as well as the value of a hepatorenal contrast 34 as illustrated, for example, in FIG. 4.

Thus, when the operator has set the first region of interest R1 in the region of the liver 21 of the B mode image, the sound speed map producer 12 produces the sound speed map of the inside of the first region of interest R1, while the kidney recognizer 10 automatically recognizes the renal cortex 23, the hepatorenal contrast calculator 13 calculates the hepatorenal contrast, and the monitor 8 displays on its screen 31 the sound speed map image 33 and the hepatorenal contrast 34 as well as the B mode image 32, enabling a high-accuracy diagnosis of the subject's liver disease.

Although the hepatorenal contrast calculator 13 calculates the average luminance B1 in the first region of interest R1 and the average luminance B2 in the second region of interest R2 based on the reception data for the B mode image stored in the reception data memory 11, the invention is not limited thereto; the average luminances B1 and B2 may be obtained based on the B mode image signal produced by the image producer 17.

Instead of the B mode image 32 and the sound speed map image 33 being displayed in juxtaposition on the screen 31 of the monitor 8, the sound speed map may be superimposed on the B mode image.

Further, not only the value of the hepatorenal contrast 34 but the average luminance B1 in the first region of interest R1 set in the liver 21 and the average luminance B2 in the second region of interest R2 set in the region of the renal cortex 23, as well as the average sound speed in the first region of interest R1 and other values may be also displayed. The value of the hepatorenal contrast 34 and the average sound speed in the first region of interest R1, for example, obtained in the past diagnosis may be displayed in the form of, for example, graph or table.

Embodiment 2

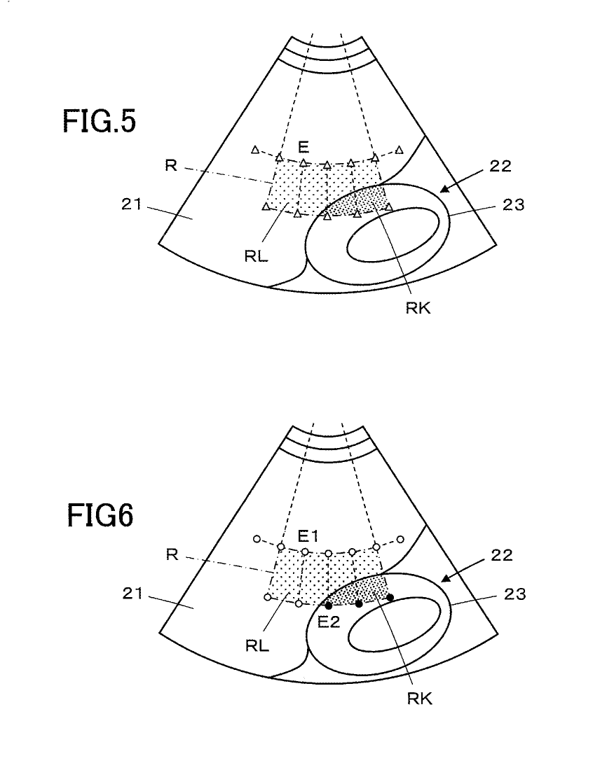

While, in the above Embodiment 1, the first region of interest R1 is set in the region of the liver 21 of the B mode image by the operation performed by the operator, and the second region of interest R2 is set in the region of the renal cortex 23 automatically recognized by the kidney recognizer 10, a single region of interest R may be set so as to extend over the liver 21 and the kidney 22 in the B mode image as illustrated in FIG. 5. The setting of the region of interest R is achieved by the operation performed by the operator through the operating unit 15.

Upon the region of interest R being set, the controller 14 sets a plurality of lattice points E indicated by “Δ” in the region of interest R and in the neighborhood thereof. Since the region of interest R are so located as to extend over the liver 21 and the kidney 22, the plurality of lattice points E are distributed over both regions, a liver region RL and a kidney region RK in the region of interest R. Thus, as illustrated in FIG. 6, the operator operates the operating unit 15 to select lattice points indicated by “∘” located in the liver region RL as lattice points E1 for obtaining information on the liver 21 and select lattice points indicated by “•” located in the kidney region RK as lattice points E2 for obtaining information on the kidney 22 from among the plurality of lattice points E.

Subsequently, the controller 14 controls the transmission circuit 2 and the reception circuit 3 to sequentially transmit and receive the ultrasonic beam with the transmission focus formed at the lattice points E1 located in the liver region RL, and the reception data produced by the reception circuit 3 for each ultrasonic beam received are sequentially stored in the reception data memory 11 as reception data for measuring the sound speed and the luminance on the liver region RL.

Similarly, the controller 14 controls the transmission circuit 2 and the reception circuit 3 to sequentially transmit and receive the ultrasonic beam with the transmission focus formed at the lattice points E2 located in the kidney region RK, and the reception data produced by the reception circuit 3 for each ultrasonic beam received are sequentially stored in the reception data memory 11 as reception data for measuring the luminance on the kidney region RK.

Thus, when the reception data for measuring the sound speed and the luminance have been obtained for all the lattice points E1 in the liver region RL, the controller 14 outputs an instruction for producing the sound speed map to the sound speed map producer 12, which then uses the reception data on the lattice points E1 stored in the reception data memory 11 to calculate local sound speeds at the lattice points E1 and produce the sound speed map of the inside of the liver region RL.

Further, upon acquisition of the reception data for measuring the luminance for all the lattice points E2 in the kidney region RK, the controller 14 instructs the hepatorenal contrast calculator 13 to calculate the hepatorenal contrast, whereupon the hepatorenal contrast calculator 13 uses the reception data on the lattice points E1 stored in the reception data memory 11 to calculate the average luminance B1 in the liver region RL and uses the reception data on the lattice points E2 stored in the reception data memory 11 to calculate the average luminance B2 in the kidney region RK, thereafter calculating the hepatorenal contrast expressed as r=B1/B2 or the ratio of the average luminance B1 in the liver region RL to the average luminance B2 in the kidney region RK.

The data on the sound speed map of the inside of the liver region RL produced by the sound speed map producer 12 and the data on the hepatorenal contrast calculated by the hepatorenal contrast calculator 13 are sent to the DSC 5 under the control by the controller 14 and undergo raster conversion through the DSC 5 as well as various image processing through the image processor 6 before being transmitted to the display controller 7. Subsequently, according to the display mode entered by the operator from the operating unit 15, the monitor 8 displays on its screen 31 the B mode image 32 and the sound speed map image 33 in juxtaposition as well as the value of the hepatorenal contrast 34 as illustrated in FIG. 4 similarly to Embodiment 1.

Thus, when the operator has set the region of interest R extending over the liver 21 and the kidney 22 in the B mode image, and the lattice points E1 for obtaining information on the liver 21 and the lattice points E2 for obtaining information on the kidney 22 have been selected, the sound speed map producer 12 produces the sound speed map of the liver region RL in the region of interest R, while the hepatorenal contrast calculator 13 calculates the hepatorenal contrast, and the monitor 8 displays the sound speed map image and the hepatorenal contrast as well as the B mode image, enabling a high-accuracy diagnosis of the subject's liver disease.

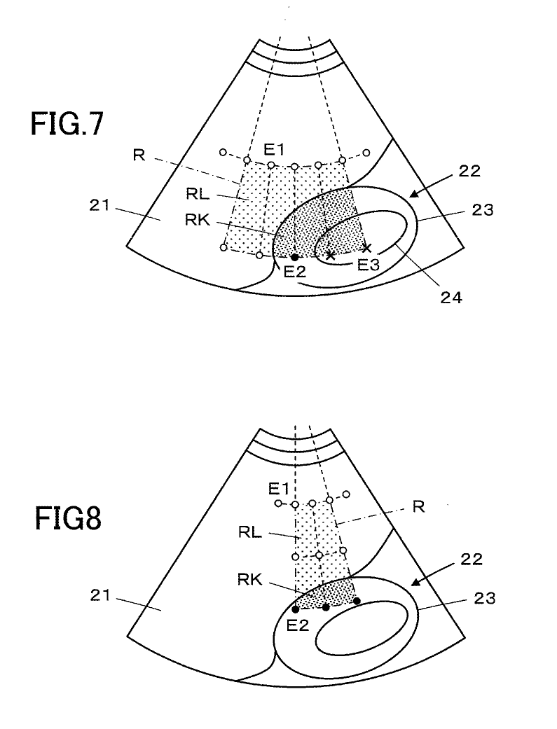

The hepatorenal contrast is preferably expressed as the ratio of an average liver luminance and an average renal cortex luminance. Therefore, where there exist among the lattice points E set by the controller 14 lattice points E3 indicated by “x” located in a kidney pelvis/kidney calyx region 24 of the kidney 22 as illustrated in FIG. 7, for example, it is desirable that the lattice points E3 are excluded from the lattice points for obtaining information on the kidney 22. That is to say, the controller 14 controls the transmission circuit 2 and the reception circuit 3 not to transmit and receive the ultrasonic beam for the lattice points E3 located in the kidney pelvis/kidney calyx region 24.

Where the region of interest R extends longer in the depth direction as illustrated in FIG. 8, a plurality of lattice points are set in the depth direction in a plurality of stages, and it is preferable to select the lattice points E1 located in the liver region RL and the lattice points E2 located in the kidney region E2 from these lattice points. Thus, a more accurate sound speed map of the inside of the liver region RL can be produced.

Embodiment 2 needs no kidney recognizer 10 illustrated in FIG. 1 since the region of interest R stretching over the liver 21 and the kidney 22 is set by the operator through the operating unit, and since the lattice points E1 in the liver region RL and the lattice points E2 in the kidney region RK are also selected by the operation through the operating unit 15.

However, when the kidney recognizer 10 automatically recognizes the kidney 22, the renal cortex 23 in particular, this enables automatic selection of the lattice points E1 located in the liver region RL and the lattice points E2 located in the kidney region RK based on the recognition results from among a plurality of lattice points E and, hence, enhanced ease of operation. Further, lattice points located in the kidney pelvis/kidney calyx region 24, such as the lattice points E3 illustrated in FIG. 7, may be automatically recognized and excluded from calculation of the average luminance B2 of the inside of the kidney region RK.

The average luminance B1 calculated using the reception data on the lattice points E1 may be an average luminance at a set sound speed that is an optimum sound speed.

Although the reception data on the lattice points E1 and E2 are stored in the reception data memory 11, the reception data on not only the lattice points E1 and E2 but points located in the periphery of the lattice points E1 and E2 may also be stored in the reception data memory 11 to calculate an optimum sound speed and an average luminance based on the reception data on the lattice points E1 and E2 and the points located in the periphery thereof.

What is claimed is:

1. An ultrasound diagnostic apparatus comprising:
a transducer array;
a transmission circuit for transmitting an ultrasonic beam from the transducer array toward a subject;
a reception circuit for processing reception signals outputted from the transducer array having received ultrasonic echoes from the subject to obtain reception data;
an image producer for producing a B mode image based on the reception data for a B mode image obtained by the reception circuit;
a monitor for displaying a B mode image produced by the image producer;
a region of interest setting unit for setting a region of interest containing at least part of a liver and at least part of a kidney of the subject in the B mode image displayed on the monitor;
a controller for controlling the transmission circuit and the reception circuit to transmit and receive ultrasonic beams with transmission focuses formed at a plurality of points set in at least a liver of the region of interest to obtain reception data for measuring a sound speed on at least the liver region of the region of interest;
a sound speed map producer for producing a sound speed map of the liver region of the region of interest based on the reception data for measuring a sound speed; and
a hepatorenal contrast calculator for calculating an average luminance in the liver region and an average luminance in the kidney region of the region of interest and calculating a hepatorenal contrast based on the calculated average luminances,
wherein the controller causes the monitor to display the sound speed map produced by the sound map producer and the hepatorenal contrast calculated by the hepatorenal contrast calculator.

2. The ultrasound diagnostic apparatus according to claim 1, further comprising a kidney recognizer for detecting the kidney based on the reception data for a B mode image.

3. The ultrasound diagnostic apparatus according to claim 2,
wherein the region of interest contains a first region of interest set in the liver on the B mode image in response to input by an operator and a second region of interest detected by the kidney recognizer,
the controller controlling the transmission circuit and the reception circuit to transmit and receive ultrasonic beams with transmission focuses formed at the plurality of points set in the first region of interest to obtain reception data for measuring a sound speed on the first region of interest,
the sound speed map producer producing a sound speed map of the inside of the first region of interest based on the reception data for measuring a sound speed on the first region of interest,
the hepatorenal contrast calculator calculating the average luminance in the first region of interest and the average luminance in the second region of interest based on the reception data for a B mode image.

4. The ultrasound diagnostic apparatus according to claim 3,
wherein the kidney recognizer detects a renal cortex of the kidney,
the second region of interest being set in the renal cortex detected by the kidney recognizer.

5. The ultrasound diagnostic apparatus according to claim 1,
wherein the region of interest is set in response to input by an operator so as to extend in both the liver and the kidney in the B mode image,
the controller setting the plurality of points as to be distributed in both the liver region and the kidney region and controlling the transmission circuit and the reception circuit to transmit and receive ultrasonic beams with transmission focuses formed at the points set in the liver region to obtain reception data for measuring a sound speed and a luminance on the liver region while transmit and receive ultrasonic beams with transmission focuses formed at the points set in the kidney region to obtain reception data for measuring a luminance on the kidney region,
the sound speed map producer producing a sound speed map of the liver region in the region of interest based on the reception data for measuring a sound speed and a luminance on the liver region,
the hepatorenal contrast calculator calculating an average luminance in the liver region of the region of interest based on the reception data for measuring a sound speed and a luminance on the liver region and calculating an average luminance in the kidney region of the region of interest based on the reception data for measuring a luminance on the kidney region.

6. The ultrasound diagnostic apparatus according to claim 5, wherein the points in the kidney region are set in a renal cortex of the kidney.

7. An ultrasound image producing method comprising the steps of:
producing a B mode image based on reception data for a B mode image obtained by transmitting and receiving ultrasonic beams through a transducer array to and from a subject to display the B mode image on a monitor;
setting a region of interest containing at least part of a liver and at least part of a kidney of the subject in the B mode image;
transmitting and receiving ultrasonic beams with transmission focuses formed at a plurality of points set in at least a liver region of the region of interest to obtain reception data for measuring a sound speed on at least the liver region of the region of interest;
producing a sound speed map of the liver region of the region of interest based on the reception data for measuring a sound speed;
calculating an average luminance in the liver region and an average luminance in the kidney region of the region of interest and calculating a hepatorenal contrast based on the calculated average luminances; and
displaying the produced sound speed map and the calculated hepatorenal contrast on the monitor.
